(19) United States
(12) Patent Application Publication
(10) Pub. No.: US 2021/0098112 A1
(43) Pub. Date: Apr. 1, 2021

(54) SYSTEM, METHOD AND/OR COMPUTER READABLE MEDIUM FOR ENHANCED PRESENTATION AND/OR INTERPRETATION OF IMAGES WITH VISUAL FEEDBACK

(71) Applicant: ClearVoxel Imaging, Inc., Waterloo (CA)

(72) Inventor: Yann Gagnon, Waterloo (CA)

(21) Appl. No.: 17/106,011

(22) Filed: Nov. 27, 2020

Related U.S. Application Data
(63) Continuation of application No. PCT/CA2019/050748, filed on May 30, 2019.
(60) Provisional application No. 62/677,894, filed on May 30, 2018.
(60) Provisional application No. 62/770,798, filed on Nov. 22, 2018.

(51) Int. Cl. G16H 30/20; G06F 3/01; G06K 9/00; G06T 7/20; G06T 7/00

(52) U.S. Cl. CPC G16H 30/20; G06F 3/013; G06K 2209/05; G06T 7/20; G06T 7/0012; G06K 9/00604

(57) ABSTRACT

The present invention is directed to systems, methods and/or computer readable media for facilitating a new medical image interpretation workflow. User actions typically are monitored using peripheral devices such as eye-trackers to infer information gleaned from the user (e.g., predicting user actions based on a pattern or detecting a visual search pattern which is inadequate). Orchestration of this information in tandem with a computer aided detection and/or diagnosis systems (“CAD”) system offers an opportunity to reduce the volume of interaction required of the user to complete the image interpretation task and/or reduce the incidence of errors during the image interpretation task.

### CROSS-REFERENCE TO RELATED APPLICATIONS

This application is a continuation of co-pending PCT Patent Application PCT/CA2019/050748, filed May 30, 2019, which claims priority to U.S. Provisional Patent Application No. 62/770,798, filed on Nov. 22, 2018 and U.S. Provisional Patent Application No. 62/677,894, filed on May 30, 2018, the entirety of all of which are hereby incorporated by reference.

### FIELD OF THE INVENTION

The present invention relates generally to methods, systems and/or computer readable media for use with images, and more specifically to methods, systems and/or computer readable media for viewing, interacting with and interpreting data including medical images.

### BACKGROUND OF THE INVENTION

Medical images are acquired and viewed in the context of the screening, diagnosis and/or monitoring of disease within various clinical settings, such as a hospital, medical imaging center or even a mobile unit. Images are read, that is viewed and interpreted, either at the same site they are acquired or transferred to be read remotely. Many types of healthcare practitioners may review and read medical imaging data, such as radiologists, primary care physicians and specialists such as cardiologists and neurologists. However, radiologists specialize in this task, which they are expected to perform with high-throughput, high-accuracy and from which they generate reports which become part of the patient health record.

In an effort to improve the efficiency and accuracy in the review of medical images, computer aided diagnostic and detection systems have been developed and commercialized in the prior art. Computer aided detection and/or diagnosis systems (“CAD systems”) detect features in images based on predefined rules, or based on models generated from training data, such as with machine learning. CAD systems may have been used by healthcare practitioners in the prior art within different paradigms, such as a concurrent reader, second reader or as an additional referral to be used in specific circumstances or when certain criteria are met.

Another area of progress in medical imaging of the prior art may relate to data acquisition itself. For example, imaging data may be acquired at high resolutions, resulting in large datasets, particularly in the case of three-dimensional images. Another instance may be variations in manipulating acquisition techniques to create new qualitative image contrasts and quantitative imaging data with more diagnostic value. Another example may be the acquisition of dynamic datasets which presents imaging data through a time-series, such as the imaging of a beating heart.

Presently, the viewing and interpretation of medical images in a clinical setting, particularly within the radiology department of a hospital, may be constrained by extreme time pressures, throughput requirements and long work hours. The interpretation of medical images in the context of screening, diagnosis and monitoring of disease is a difficult visuo-cognitive task requiring a high level of training and experience. Additionally, the trends towards larger and more complex imaging datasets, combined with the inclusion of CAD systems, have combined to create burdensome forces on the workflow of those who review and report on medical imaging in high volumes, namely radiologists. In particular, radiologists may not be able to adopt new technologies if they decrease their throughput by necessitating additional action on their part, including accessing these technologies through another software package or computer application. In fact, due to the intensive workloads and an existing intensive requirement of computer interactions and manual input, any additional burden becomes a barrier in terms of the adoption of these technologies by radiologists.

Attempts to overcome the problems of the prior art may have involved large medical imaging equipment and information technology companies providing some level of integration across their technologies, often within an ecosystem that is specific to their commercial offerings. The integration is to benefit the installation from an information technology perspective and for the workflow of the radiologist.

Moreover, smaller providers of medical imaging technology may have offered specialized computer software that provide little opportunity for integration. Technologies may have been bundled and offered within a stand-alone application package and will instead offer provisions for interoperability, such as the ability to retrieve or send imaging data to an existing archiving system.

These prior art integration offerings from large medical equipment and information technology companies may have been limited and may not have been introduced at a rate that serves the speed of innovation. In particular, the bulk of innovation may have been innovations in software, as opposed to hardware. In addition, individual healthcare providers may not retain complete control on which technologies they are able to adopt because they are limited to the options as made available within a specific company's commercial ecosystem. The stand-alone offerings from smaller companies present a barrier to the adoption of new technologies because they add a burden to the workflow of radiologists.

None of the previous solutions may be able to effectively mitigate or even reduce the level of manual user interaction inflicted upon radiologists. Any additional functionality, even if fully integrated from a software perspective, may result in additional button clicks/activations or menu item selections. Further, the results from advanced analyses must typically be retrieved, processed, acknowledged, or dismissed manually. Additionally, humans may be imperfect at the interpretation of medical images. Even expert radiologists may be subject to the same faults in visual attention as non-expert users in their everyday tasks. CAD systems in the prior at may not be able to mitigate this effect in practice, which may be due to an inability to optimize the interaction between these systems and humans.

Overall, prior art solutions may be burdensome within the reality of clinical context of healthcare providers and may not provide the value required to offset this burden.

What may be needed is a method and system to implement a new medical image reading workflow which enables the use of additional technologies without introducing additional burdens on the user workflow of the healthcare practitioner performing this task.

It is an object of the present invention to obviate or mitigate one or more of the aforementioned disadvantages and/or shortcomings associated with the prior art, to provide one of the aforementioned needs or advantages, and/or to achieve one or more of the aforementioned objects of the invention.

### SUMMARY OF THE INVENTION

In view of the potential limitations inherent in the prior art for viewing and/or interpreting images (e.g., medical images), the present disclosure provides a system, method and/or computer readable medium for the enhanced viewing and interpretation of such images.

According to an aspect of the invention, there is disclosed a system for providing visual feedback of image data to a user. The system includes a workflow environment having an imaging display for presenting content to the user, a biometric interaction system operative to facilitate interaction with the imaging display by the user and a computer-aided diagnosis system. The biometric interaction system includes: (i) a motion tracking device for receiving motion data associated with a movement of the user; (ii) an eye-tracking device for receiving gaze data associated with an eye gaze of the user; and (iii) a peripheral processor operative to collect and transmit the motion data and the eye gaze data. The computer-aided diagnosis system includes a system processor operative to: (i) electronically receive the motion data and the gaze data from the peripheral processor; (ii) analyze the image data using a computer-aided diagnosis algorithm to automatically identify a feature associated with the image data; (iii) present the image data and/or the identified feature to the user on the imaging display; and (iv) automatically apply the motion data to the imaging display using a gesture algorithm and the gaze data to the imaging display using an eye tracking analysis module algorithm to manipulate the content. Thus, according to the invention, the system is operative to facilitate enhanced viewing and/or interpretation of the image data by the user.

According to an aspect of one preferred embodiment of the invention, the imaging display of the system may preferably, but need not necessarily, include a primary imaging display and a secondary imaging display.

According to an aspect of one preferred embodiment of the invention, the system may preferably, but need not necessarily, include a database to electronically store the motion data, the gaze data, the image data, and/or the identified feature.

According to an aspect of one preferred embodiment of the invention, the system may preferably, but need not necessarily, include one or more predetermined workflows to facilitate the provision of visual feedback of the image data by the user.

According to an aspect of one preferred embodiment of the invention, the workflows may preferably, but need not necessarily, include an imaging workflow, an alternate views workflow, a reporting workflow, a worklist workflow and/or a CAD workflow.

According to an aspect of one preferred embodiment of the invention, the biometric interaction system may preferably, but need not necessarily, include an array of interdependent devices.

According to an aspect of one preferred embodiment of the invention, the array of interdependent devices may preferably, but need not necessarily, include two or more eye-tracking devices.

According to the invention, there is provided a method for providing visual feedback of image data to a user in a workflow environment including an imaging display for presenting content to the user. The method includes: (a) a step of operating a biometric interaction system to facilitate interaction with the imaging display by the user, the biometric interaction system including: (i) a motion tracking device adapted to receive motion data associated with a movement of the user, (ii) an eye-tracking device adapted to receive gaze data associated with an eye gaze of the user, and (iii) a peripheral processor to collect and transmit the motion data and the eye gaze data; and (b) a step of operating a computer-aided diagnosis system including a system processor to: (i) electronically receive the motion data and the gaze data from the peripheral processor, (ii) analyze the image data using a computer-aided diagnosis algorithm to automatically identify a feature associated with the image data, (iii) present the image data and/or the identified feature to the user on the imaging display, and (iv) automatically apply the motion data to the imaging display using a gesture algorithm and the gaze data to the imaging display using an eye tracking analysis module algorithm to manipulate the content. Thus, according to the invention, the method is operative to facilitate enhanced viewing and/or interpretation of the image data by the user.

According to an aspect of one preferred embodiment of the invention, the imaging display of the method may preferably, but need not necessarily, include a primary imaging display and a secondary imaging display.

According to an aspect of one preferred embodiment of the invention, the method may preferably, but need not necessarily, include a step of electronically storing the motion data, the gaze data, the image data and/or the identified feature in a database.

According to an aspect of one preferred embodiment of the invention, the method may preferably, but need not necessarily, include a step of applying one or more predetermined workflows to facilitate the provision of visual feedback of the image data by the user.

According to an aspect of one preferred embodiment of the invention, the one or more predetermined workflows applied in the method may preferably, but need not necessarily, include an imaging workflow, an alternate views workflow, a reporting workflow, a worklist workflow and/or a CAD workflow.

According to an aspect of one preferred embodiment of the invention, the biometric interaction system of the method further includes an array of interdependent devices.

According to an aspect of one preferred embodiment of the invention, the array of interdependent devices used in the method may preferably, but need not necessarily, include two or more eye-tracking devices.

According to the invention, there is provided a non-transitory computer readable medium on which is physically stored executable instructions, which upon execution, will provide visual feedback of image data to a user within a workflow environment including an imaging display for presenting content to the user, a biometric interaction system including a motion tracking device for receiving motion data of the user and an eye-tracking device for receiving gaze data of the user to facilitate interaction with the imaging display by the user and a computer-aided diagnosis system. The executable instructions include processor instructions for a peripheral processor and/or a system processor to automatically: (a) collect and/or electronically communicate the motion data from the peripheral processor to the system processor; (b) collect and/or electronically communicate the gaze data from the peripheral processor to the system processor; (c) automatically identify a feature associated with the image data using a computer-aided diagnosis algorithm; (d) automatically present the image data and/or the identified feature to the user on the imaging display; and (e) automatically manipulate the content of the imaging display using a gesture algorithm on the motion data and the eye tracking analysis module algorithm on the gaze data. Thus, according to the invention, the computer readable medium is operative to facilitate enhanced viewing and/or interpretation of the image data by the user.

Persons of ordinary skill in the art may appreciate that new medical imaging technologies, especially those introduced within the scope of the radiological workflow environment and/or of a CAD system may be destined to place additional demands on a user. It may have been reported that the already fast-paced demands of clinical throughput placed on radiologists make the adoption of these new technologies difficult. Accordingly, even though a new technology is intended to offer a significant benefit in certain scenarios, the negative impact on the productivity of the radiologist could limit its adoption. In accordance with a preferred embodiment of the present invention, the inclusion of a biometric interaction system preferably removes this limitation. A workflow instructor preferably reduces and/or streamlines the interactions required of the user. Further, within a preferred embodiment of the present invention, components of the biometric interaction system such as an eye gaze tracking and analysis module may be adapted to train the workflow instructor for improved performance, such as with the use of a user eye gaze model.

In accordance with a preferred embodiment of the present invention, there may be provided mitigation of interruptions and distractions (or other interactions that can affect cognitive function during the task and affect the observations and conclusion of the radiologist) to which radiologists are often subjected. In a preferred embodiment, the biometric interaction system may detect an interruption which takes a user's attention away from the radiological workflow or the CAD system. The biometric interaction system preferably detects when the user returns their attention to the previous task. By collecting and storing information about the user's activities, such as gaze patterns, before the interruption and making available graphical, auditory or other features which are indicative of the user's prior state of attention, the effect of the interruption may be mitigated. In accordance with a preferred embodiment, one such example may be the implementation of an imaging bookmark within a viewing area to indicate to the user which areas of an image or graphics user interface their attention was focused on before the interruption.

In accordance with a preferred embodiment of the present invention, there may be provided an ability to render sensitive or confidential medical information invisible to any person without appropriate credentials. In a preferred embodiment, for example, if a radiologist leaves the radiology workflow environment, the workflow instructor may detect the absence of the authorized user and blur or remove information previously presented on the screen. In a preferred embodiment, a workstation may selectively block certain and/or predetermined information at the radiology workstation while enabling the user to perform predetermined functions which do not infringe of confidentiality and/or security requirements.

In accordance with one or more preferred embodiments, the system, method and/or computer readable medium of the present invention may ease the burden of manual interaction imposed by the heavy demands of the radiological workflow while simultaneously allowing the contextual insertion of new image analysis technologies. This may preferably but need not necessarily result in both an increased clinical productivity and/or clinical utility to the medical imaging scenario, in contrast to the traditional compromise between the two.

In accordance with one or more preferred embodiments, the system, method and/or computer readable medium of the present invention may provide for a radiologist to self-audit his or her observations and/or conclusions with respect to the medical imaging data he or she is interpreting.

According to an aspect of the present invention, there is preferably disclosed a system for enhanced viewing and/or interpretation of image data with visual feedback by a user. The system may preferably include: a workflow environment for viewing the image data by the user; a biometric interaction system to facilitate interaction with the image data by the user; and a computer-aided diagnosis subsystem for detecting a feature in the image data by the user.

According to an aspect of the present invention, there is preferably disclosed a method for enhanced viewing and/or interpretation of image data with visual feedback by a user.

The method may preferably include: providing a workflow environment for viewing the image data by the user; providing a biometric interaction system to facilitate interaction with the image data by the user; and providing a computer-aided diagnosis subsystem for detecting a feature in the image data by the user.

According to an aspect of the present invention, there is preferably disclosed a non-transitory computer readable medium encoded with executable instructions for enhanced viewing and/or interpretation of image with visual feedback data by a user. The non-transitory computer readable medium may preferably include: providing a workflow environment for viewing the image data by the user; providing a biometric interaction system to facilitate interaction with the image data by the user; and providing a computer-aided diagnosis subsystem for detecting a feature in the image data by the user.

Alterations or modifications of the present invention as described for specific types of medical imaging, imaging data, or clinical scenarios are understood to be within the scope of the present invention.

Other advantages, features and characteristics of the present invention, as well as methods of operation and functions of the related features of the system, method, device and computer readable medium, and the combination of steps, parts and economies of manufacture, will become more apparent upon consideration of the following detailed description and the appended claims with reference to the accompanying drawings, the latter of which are briefly described herein below.

### BRIEF DESCRIPTION OF THE DRAWINGS

The novel features which are believed to be characteristic of the system, device, method and/or computer readable medium according to the present invention, as to their structure, organization, use, and method of operation, together with further objectives and advantages thereof, will be better understood from the following drawings in which presently preferred embodiments of the invention will now be illustrated by way of example. It is expressly understood, however, that the drawings are for the purpose of illustration and description only, and are not intended as a definition of the limits of the invention. In the accompanying drawings:

FIG. 1 is a schematic diagram of a radiological workflow environment, a biometric interaction system, and a CAD system according to a preferred embodiment of the present invention;

FIG. 2 is an illustration of the radiological workflow environment according to a preferred embodiment of the present invention;

FIG. 3 is an illustration of the imaging workflow according to a preferred embodiment of the present invention;

FIG. 4 is an illustration of alternate views of the imaging workflow according to a preferred embodiment of the present invention;

FIG. 5 is an illustration of the reporting workflow according to a preferred embodiment of the present invention;

FIG. 6 is an illustration of the worklist workflow according to a preferred embodiment of the present invention;

FIG. 7 is an illustration of the CAD workflow according to a preferred embodiment of the present invention;

FIG. 8 is an illustration of the imaging bookmark according to a preferred embodiment of the present invention;

FIG. 9 is a schematic diagram of the gaze analysis according to a preferred embodiment of the present invention;

FIG. 10 is a schematic diagram of the gaze-tracking apparatus in a multi-screen radiology workflow environment according to a preferred embodiment of the present invention; and

FIG. 11 is a schematic diagram of a cascading tracking apparatus with supplementary tracking elements according to a preferred embodiment of the present invention.

### DETAILED DESCRIPTION OF THE PREFERRED EMBODIMENTS

The description that follows, and the embodiments described therein, may be provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order to more clearly depict certain embodiments and features of the invention.

The present disclosure may be described herein with reference to system architecture, block diagrams and flowchart illustrations of methods, and computer program products according to various aspects of the present disclosure. It may be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flow diagram illustrations support combinations of means for performing the specified functions, combinations of steps for performing the specified functions, and program instruction means for performing the specified functions. It may also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

The present disclosure may now be described in terms of an exemplary system in which the present disclosure, in various embodiments, may be implemented. This may be for convenience only and may be not intended to limit the application of the present disclosure. It may be apparent to one skilled in the relevant art(s) how to implement the present disclosure in alternative embodiments.

In this disclosure, a number of terms and abbreviations may be used. The following definitions and descriptions of such terms and abbreviations are provided in greater detail.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

As used herein, a person skilled in the relevant art may generally understand the term “interactable” to generally mean interaction with an object (e.g., an image presented on a graphical user interface).

It should also be appreciated that the present invention can be implemented in numerous ways, including as a system, method, and/or a computer readable medium wherein program instructions are sent over a network (e.g., optical or electronic communication links). In this specification, these implementations, or any other form that the invention may take, may be referred to as processes or methods. In general, the order of the steps of the disclosed processes may be altered within the scope of the invention.

Preferred embodiments of the present invention can be implemented in numerous configurations depending on implementation choices based upon the principles described herein. Various specific aspects are disclosed, which are illustrative embodiments not to be construed as limiting the scope of the disclosure. Although the present specification describes components and functions implemented in the embodiments with reference to standards and protocols known to a person skilled in the art, the present disclosures as well as the embodiments of the present invention are not limited to any specific standard or protocol. Each of the standards for non-mobile and mobile computing, including the Internet and other forms of computer network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same functions are considered equivalents.

As those of ordinary skill in the art would generally understand, the Internet is a global computer network which comprises a vast number of computers and computer networks which are interconnected through communication links. A person skilled in the relevant art may understand that an electronic communications network of the present invention, may include, but is not limited to, one or more of the following: a local area network, a wide area network, peer-to-peer communication, an intranet, or the Internet. The interconnected computers exchange information using various services, including, but not limited to, electronic mail, Gopher, web-services, application programming interface (API), File Transfer Protocol (FTP). This network allows a server computer system (a Web server) to send graphical Web pages of information to a remote client computer system. The remote client computer system can then display the Web pages via its web browser. Each Web page (or link) of the “world wide web” (“WWW”) is uniquely identifiable by a Uniform Resource Locator (URL). To view a specific Web page, a client computer system specifies the URL for that Web page in a request (e.g., a HyperText Transfer Protocol (“HTTP”) request). The request is forwarded to the Web server that supports the Web page. When the Web server receives the request, it sends the Web page to the client computer system. When the client computer system receives the Web page, it typically displays the Web page using a browser. A web browser or a browser is a special-purpose application program that effects the requesting of web pages and the displaying of web pages and the use of web-based applications. Commercially available browsers include Microsoft Internet Explorer and Firefox, Google Chrome among others. It may be understood that with embodiments of the present invention, any browser would be suitable.

Web pages are typically defined using HTML. HTML provides a standard set of tags that define how a Web page is to be displayed. When a provider indicates to the browser to display a Web page, the browser sends a request to the server computer system to transfer to the client computer system an HTML document that defines the Web page. When the requested HTML document is received by the client computer system, the browser displays the Web page as defined by the HTML document. The HTML document contains various tags that control the displaying of text, graphics, controls, and other features. The HTML document may contain URLs of other Web pages available on that server computer system or other server computer systems.

A person skilled in the relevant art may generally understand a web-based application refers to any program that is accessed over a network connection using HTTP, rather than existing within a device's memory. Web-based applications often run inside a web browser or web portal. Web-based applications also may be client-based, where a small part of the program is downloaded to a user's desktop, but processing is done over the Internet on an external server. Web-based applications may also be dedicated programs installed on an internet-ready device, such as a smart phone or tablet. A person skilled in the relevant art may understand that a web site may also act as a web portal. A web portal may be a web site that provides a variety of services to users via a collection of web sites or web-based applications. A portal is most often one specially designed site or application that brings information together from diverse sources in a uniform way. Usually, each information source gets its dedicated area on the page for displaying information (a portlet); often, the user can configure which ones to display. Portals typically provide an opportunity for users to input information into a system. Variants of portals include “dashboards”. The extent to which content is displayed in a “uniform way” may depend on the intended user and the intended purpose, as well as the diversity of the content. Very often design emphasis is on a certain “metaphor” for configuring and customizing the presentation of the content and the chosen implementation framework and/or code libraries. In addition, the role of the user in an organization may determine which content can be added to the portal or deleted from the portal configuration.

It may be generally understood by a person skilled in the relevant art that the term “mobile device” or “portable device” refers to any portable electronic device that can be used to access a computer network such as, for example, the internet. Typically, a portable electronic device comprises a display screen, at least one input/output device, a processor, memory, a power module and a tactile man-machine interface as well as other components that are common to portable electronic devices individuals or members carry with them on a daily basis. Examples of portable devices suitable for use with the present invention include, but are not limited to, smart phones, cell phones, wireless data/email devices, tablets, PDAs, and MP3 players, etc.

It may be generally understood by a person skilled in the relevant art that the term “network ready device” or “internet ready device” refers to devices that are capable of connecting to and accessing a computer network, such as, for example, the Internet, including but not limited to an IoT device. A network ready device may assess the computer network through well-known methods, including, for example, a web-browser. Examples of internet-ready devices include, but are not limited to, mobile devices (including smart-phones, tablets, PDAs, etc.), gaming consoles, and smart-TVs. It may be understood by a person skilled in the relevant art that embodiment of the present invention may be expanded to include applications for use on a network ready device (e.g., cellphone). In a preferred embodiment, the network ready device version of the applicable software may have a similar look and feel as a browser version but that may be optimized to the device. It may be understood that other “smart” devices (devices that are capable of connecting to and accessing a computer network, such as, for example, the internet) such as sensors or actuators, including but not limited to smart valves, smart lights, IoT devices, etc.

It may be further generally understood by a person skilled in the relevant art that the term “downloading” refers to receiving datum or data to a local system (e.g., mobile device) from a remote system (e.g., a client) or to initiate such a datum or data transfer. Examples of a remote systems or clients from which a download might be performed include, but are not limited to, web servers, FTP servers, email servers, or other similar systems. A download can mean either any file that may be offered for downloading or that has been downloaded, or the process of receiving such a file. A person skilled in the relevant art may understand the inverse operation, namely sending of data from a local system (e.g., mobile device) to a remote system (e.g., a database) may be referred to as “uploading”. The data and/or information used according to the present invention may be updated constantly, hourly, daily, weekly, monthly, yearly, etc. depending on the type of data and/or the level of importance inherent in, and/or assigned to, each type of data. Some of the data may preferably be downloaded from the Internet, by satellite networks or other wired or wireless networks.

Features of the present invention may be implemented with computer systems which are well known in the art. Generally speaking, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (“BIOS”) stored in a read only memory (“ROM”) and one or more program modules such as operating systems, application programs and program data stored in random access memory (“RAM”). Computers may also include a variety of interface units and drives for reading and writing data. A user of the system can interact with the computer using a variety of input devices, all of which are known to a person skilled in the relevant art.

One skilled in the relevant art would appreciate that the device connections mentioned herein are for illustration purposes only and that any number of possible configurations and selection of peripheral devices could be coupled to the computer system.

Computers can operate in a networked environment using logical connections to one or more remote computers or other devices, such as a server, a router, a network personal computer, a peer device or other common network node, a wireless telephone or wireless personal digital assistant. The computer of the present invention may include a network interface that couples the system bus to a local area network (“LAN”). Networking environments are commonplace in offices, enterprise-wide computer networks and home computer systems. A wide area network (“WAN”), such as the Internet, can also be accessed by the computer or mobile device.

It may be appreciated that the type of connections contemplated herein are exemplary and other ways of establishing a communications link between computers may be used in accordance with the present invention, including, for example, mobile devices and networks. The existence of any of various well-known protocols, such as TCP/IP, Frame Relay, Ethernet, FTP, HTTP and the like, may be presumed, and computer can be operated in a client-server configuration to permit a user to retrieve and send data to and from a web-based server. Furthermore, any of various conventional web browsers can be used to display and manipulate data in association with a web-based application.

The operation of the network ready device (i.e., a mobile device) may be controlled by a variety of different program modules, engines, etc. Examples of program modules are routines, algorithms, programs, objects, components, data structures, etc. that perform particular tasks or implement particular abstract data types. It may be understood that the present invention may also be practiced with other computer system configurations, including multiprocessor systems, microprocessor-based or programmable consumer electronics, network PCS, personal computers, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Features of the present invention may be implemented with an IoT network that includes various devices (including IoT devices) and/or other physical objects. For example, in various embodiments, the devices and/or other physical objects in the IoT network may include, among other things, one or more IoT devices having communication capabilities, non-IoT devices having communication capabilities, and/or other physical objects that do not have communication capabilities.

Features of the present invention may be implemented on a Blockchain which is a peer-to-peer decentralized open ledger, and may rely on a distributed network shared between its users where everyone holds a public ledger of every transaction carried out using the architecture, which are then checked against one another to ensure accuracy, preferably using one of a variety of cryptographic functions. This ledger is called the “blockchain”. Blockchain may be used instead of a centralized third-party auditing and being responsible for transactions. The blockchain is a public ledger that records transactions. A novel solution accomplishes this without any trusted central authority: maintenance of the blockchain is performed by a peer-to-peer network of communicating nodes running software. Network nodes can validate transactions, add them to their copy of the ledger, and then broadcast these ledger additions to other nodes. The blockchain is a distributed database; in order to independently verify the chain of ownership or validity of any and every transaction, each network node stores its own copy of the blockchain.

Embodiments of the present invention may implement Artificial Intelligence (“AI”) or machine learning (“ML”) algorithms. AI and ML algorithms are general classes of algorithms used by a computer to recognize patterns and may include on or more of the following individual algorithms: nearest neighbor, naive Bayes, decision trees, linear regression, principle component analysis (“PCA”), support vector machines (“SVM”), evolutionary algorithms, and neural networks. These algorithms may “learn” or associate patterns with certain responses in several fashions, including: supervised learning, unsupervised learning, semi-supervised learning, and reinforcement learning.

Embodiments of the present invention can be implemented by a software program for processing data through a computer system. It may be understood by a person skilled in the relevant art that the computer system can be a personal computer, mobile device, notebook computer, server computer, mainframe, networked computer (e.g., router), workstation, and the like. In one embodiment, the computer system includes a processor coupled to a bus and memory storage coupled to the bus. The memory storage can be volatile or non-volatile (i.e. transitory or non-transitory) and can include removable storage media. The computer can also include a display, provision for data input and output, etc. as may be understood by a person skilled in the relevant art.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. is here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

In accordance with a preferred aspect of the present invention, a person skilled in the relevant art would generally understand the term “application” or “application software” to refer to a program or group of programs designed for end users. While there are system software, typically but not limited to, lower level programs (e.g. interact with computers at a basic level), application software resides above system software and may include, but is not limited to database programs, word processors, spreadsheets, etc. Application software may be grouped along with system software or published alone. Application software may simply be referred to as an “application.”

It should be borne in mind, however, that all of these and similar terms are to be associated with the appropriate physical quantities and are merely convenient labels applied to these quantities. Unless specifically stated otherwise as apparent from the following discussions, it is appreciated that throughout the present invention, discussions utilizing terms such as “receiving”, “creating”, “providing”, “communicating” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices.

In a preferred embodiment, the system, method and/or computer readable medium of the present invention includes viewing and interpreting image data (e.g., medical images in a diagnostic context of a healthcare setting such as a hospital, office or clinic), methods of displaying and navigating image data (e.g., medical images and medical image-related data) that is the result of computer analysis using algorithms for image interpretation, including but not limited to those provided by computer-aided detection and diagnosis systems (alternately “CAD systems”) used for the viewing and interpretation of image data (e.g., data including medical images). Additional preferable embodiments include additional functions for viewing and interpreting of image data (e.g., data including medical images) such as user interaction with software adapted for reporting, logistical support and/or retrieval of data (e.g., data including patient information).

Embodiments of the present invention provide a system, method and/or computer readable medium for viewing and/or interpreting image data which preferably retains all the features of the systems which exist in the prior art while offering additional features and functionality.

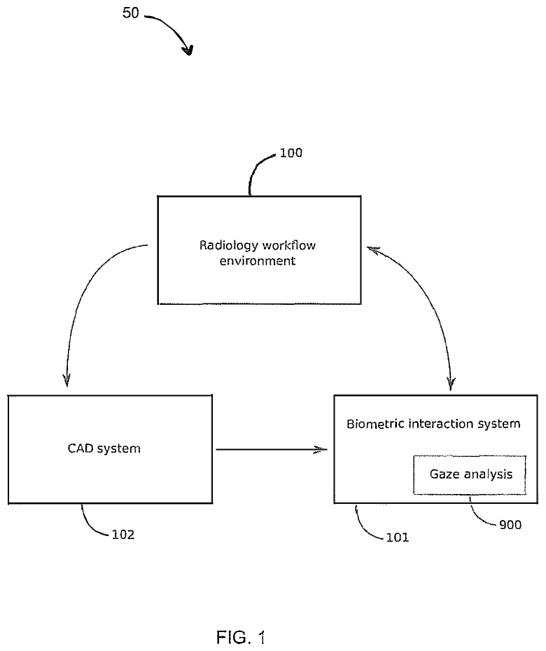

In a preferred embodiment, and as depicted in FIG. 1, the system 50 of the present invention is provided with a radiology workflow environment 100 (alternatively “radiological workflow environment 100”), a CAD system 102, and/or a biometric interaction system 101. In preferable embodiments, the radiological workflow environment 100 operates in a traditional manner (e.g., as disclosed in the prior art) with augmentation of the function of the radiological workflow environment 100 by interactions with the CAD system 102 and/or the biometric interaction system 101. The CAD system 102 preferably includes: a traditional CAD system as found in the prior art; CAD systems that provide quantitative and/or qualitative data/insights based on imaging or other acquired data (e.g, in a medical clinic); and/or a CAD system which has been optimized for use with the biometric interaction system 101. The biometric interaction system 101 preferably includes hardware and/or software adapted to interpret input from a user, whether voluntary or involuntary. In preferable embodiments, the biometric interaction system 101 includes a combination of one or more motion trackers and/or one or more sensors including but not limited to an eye-tracking device (e.g., gaze analysis 900), cameras of various spectral capabilities (e.g., visible spectrum, infrared radiation, etc.), motion or gesture control devices, electroencephalography (“EEG”) reading device, brain machine interface, or other.

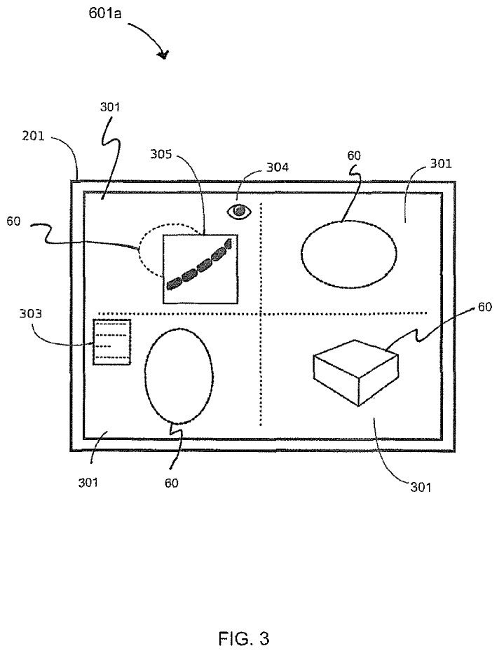

FIGS. 3 to 7 depict various workflows 601a-f that preferably, in whole or in part, make up a complete workflow 601 for use by the user 210 for viewing and/or interpreting images and/or image data 60 (e.g., medical images and/or data including medical images) (the reference 601c is not used in the figures). Persons of skill in the art will appreciate that the addition and/or combination of the CAD system 102 and the biometric interaction system 101 may include a system and/or may facilitate methods for viewing, interacting with, and/or interpreting image data 60 including images. As used herein, “images”, “image data,” and “data including images” may be used interchangeably.

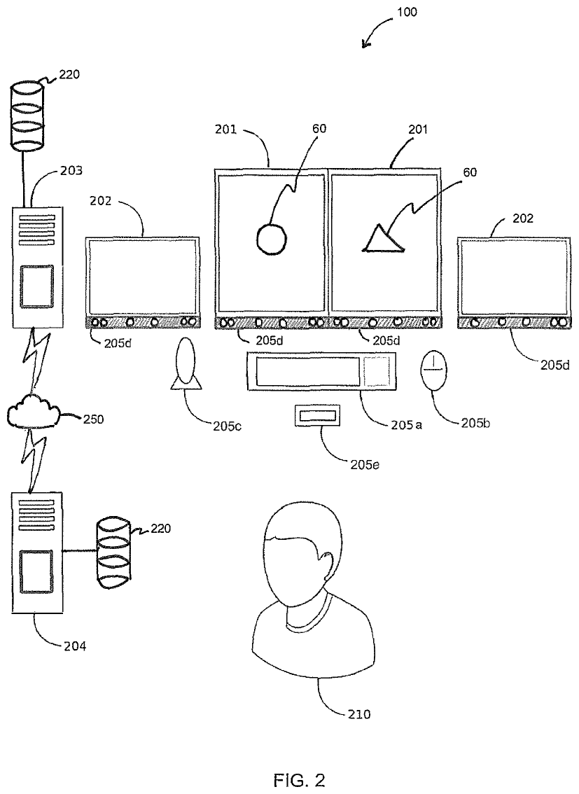

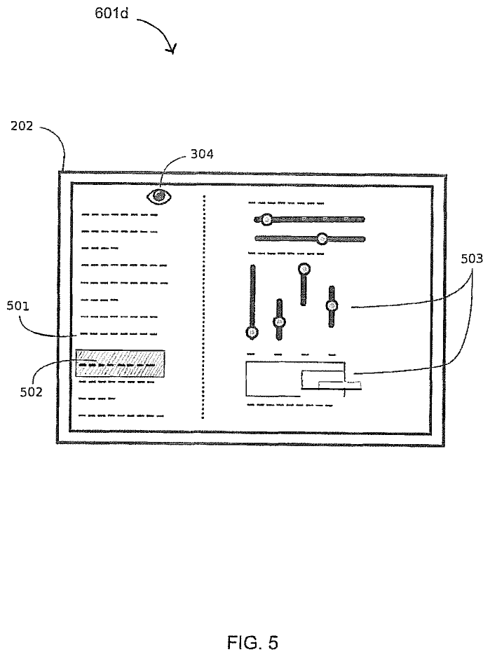

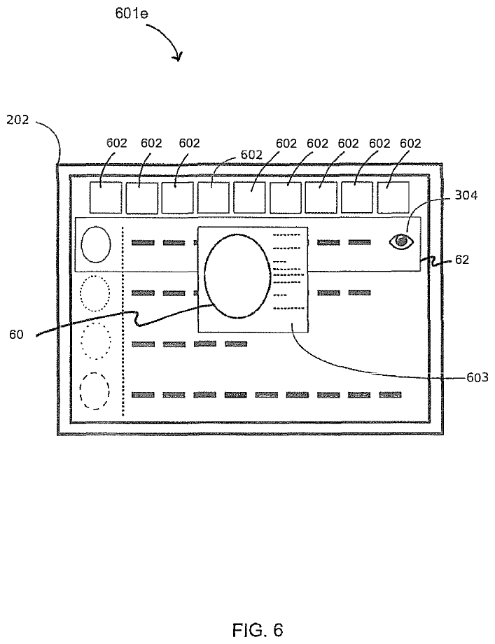

Features of a radiological workflow environment 100 in accordance with a preferred embodiment of the present invention are depicted in FIG. 2 including one or more graphical user interfaces (“GUIs” alternately “imaging display”) which may preferably be adapted to include one or more primary imaging displays 201 and one or more secondary workflow displays 202. Preferably, the one or more primary imaging displays 201 are optimized for viewing image data 60 (e.g., data including medical images), in grayscale or in color, including visualization of an output from a CAD system 102. The one or more secondary workflow displays 202 are preferably optimized for presenting workflow features such as, but not limited to, those described in FIGS. 5 and 6. The workflow environment 100 preferably includes a local processor 203 and/or a remote processor 204 (e.g., as provided in a computer system) in communication with the one or more primary imaging displays 201 and/or the one or more secondary workflow displays 202 via electronic means (e.g., a computer system) and/or a communications network 250. In some preferred embodiments, the one or more primary imaging displays 201 and/or the one or more secondary workflow displays 202 are adapted to include local processor 203 and additional computing components (e.g., memory, database, etc.) of a computer system to facilitate implementation of the embodiments of the present invention at the display level only. The processors 203, 204 are preferably used to implement the functionalities in accordance with an embodiment of the present invention.

In some preferable embodiments, the user 210 is a radiologist, technician, physician, or other clinical staff. The user 210 preferably interacts with the radiology workflow environment 100, the CAD system 102 and/or the biometric interaction system 101 via a set of peripherals (alternately, “input/output devices” or “I/O devices”) which preferably include, but are not limited to, a keyboard 205a, a computer mouse 205b, voice-operated dictation and a multi-function device 205c (e.g., a dictaphone with configurable buttons such as the PowerMic offered by Nuance), an eye-tracking device 205d (e.g., eye tracking glasses or screen-based eye trackers such as those offered by EyeTechDS and Tobii), and/or a motion tracking device 205e. The motion tracking device 205e is preferably adapted to include gesture-tracking. The keyboard 205a, a computer mouse 205b, voice-operated dictation and a multi-function device 205c, an eye-tracking device 205d, and/or a motion tracking device 205e are collectively referred to as “peripherals 205”. Persons skilled in the art may appreciate that different embodiments of the present invention may be implemented with different combinations or pluralities of peripherals 205.

Persons having skill in the art will appreciate that eye tracking is the process of measuring either the point of gaze (i.e., where one is looking) or the motion of an eye relative to the head. An eye tracker is preferably a device for measuring eye positions and eye movement. Some methods for measuring eye movement include, but are not limited to, the use of video images from which the eye position is extracted. Eye-trackers preferably measure rotations of the eye, including: (i) measurement of the movement of an object (e.g., a special contact lens) attached to the eye; (ii) optical tracking without direct contact to the eye (e.g., tracking of light reflected from the eye using a camera, tracking features from inside the eye such as retinal blood vessels, etc.); and/or (iii) measurement of electric potentials using electrodes placed around the eyes.

Persons skilled in the art will appreciate that motion tracking includes motion capture and is the process of recording the movement of objects or people. Optical systems preferably utilize data captured from image sensors to triangulate the three-dimensional position of a user between one or more cameras calibrated to provide overlapping projections. Data acquisition may be implemented using special markers (e.g., semi-passive markers, passive markers, active markers, etc.) associated with a user. Marker systems produce data with three degrees of freedom for each marker, and rotational information is determined from the relative orientation of three or more markers (e.g., shoulder, elbow and wrist markers provide the angle of the elbow). Motion capture devices may also include a markerless approach that do not require users to wear markers for tracking. Motion capture algorithms (including machine learning algorithms) preferably analyze optical input of the user to identify human forms, breaking them down into constituent parts for tracking. Motion capture devices preferably include an optical imaging system, a mechanical tracking platform, and a tracking processor. The optimal imaging system is preferably adapted to convert the light from a target area into a digital image the tracking processor can process. The mechanical tracking platform is preferably associated with the optical imaging system and is adapted to manipulate the optical imaging system so that it always points to the target being tracked. The tracking processor (which may be the local processor 203 and/or the remote processor 204) is preferably adapted to capture images from the optical imaging system, analyze the image to extract target position and control the mechanical tracking platform to follow the target. In an alternate embodiment, motion tracking includes non-optical systems such as inertial systems, mechanical motion and/or magnetic systems).

Persons skilled in the art will appreciate that gesture tracking or gesture recognition includes the interpretation of human gestures using gesture algorithms. Gestures can originate from any bodily motion or state but commonly originate from the face or hand. Users can use simple gestures to control or interact with devices without physically touching them. The ability to track a person's movements and determine what gestures they may be performing can be achieved through various tools including, but not limited to, gloves (i.e., adapted to provide input about the position and rotation of the hands using magnetic or inertial tracking devices), depth-aware cameras (i.e., specialized cameras such as structured light or time-of-flight cameras to generate a depth map of what is being seen through the camera at a short range, and use of this data to approximate a three-dimensional representation of what is being seen), single cameras, stereo cameras (i.e., two cameras whose relations to one another are known, a three-dimensional representation can be approximated by the output of the cameras), and/or gesture-based controllers (i.e., controllers act as an extension of the body so that when gestures are performed, some of their motion can be captured by software).

In a preferable embodiment, the user 210 utilizes and interacts with a specific workflow 601, such as those depicted in FIGS. 3 to 8 via, for example, the peripherals 205. In one embodiment the workflow 601 is an imaging workflow 601a including one or more viewing panes 301. In a preferred embodiment, the viewing panes 301 are adapted to display two-dimensional images, three-dimensional images, or any extension thereof including a time-series (for example, videos comprising a plurality of successive two-dimensional images or three-dimensional images) or data providing additional dimensionality. The user interacts with the components of the imaging workflow 601a via traditional means, as in the prior art, and/or preferably via the biometric interaction system 101. In preferable embodiments of the present invention, the user interacts with the eye-tracking device 205d and/or the motion tracking device 205e (e.g., to track one or more gestures of the user) to interact with and/or instruct the various components of the imaging workflow 601a displayed on the viewing panes 301, including the display of text information 303 (alternately “text data 303”), the manipulation and use of a magnification area 305 and/or the dynamic navigation of the imaging data 60 in any of the viewing panes 301. In preferable embodiments of the present invention, an indicator 304 is displayed in one or more of the viewing panes 301 (i.e., at any appropriate or predetermined area within the imaging workflow 601a and/or any other workflows 601) to render the user 210 aware of any relevant and/or desired state or states. For example, the indicator 304 may be an eye-gaze indicator to facilitate awareness by the system or an alert or indication of whether a component of the imaging workflow 601a is currently enabled or activated by the user's gaze. The indicator 304 may be a gesture tracking indicator or be adapted to provide an indication of a state of any component of the biometric interaction system in a workflow.

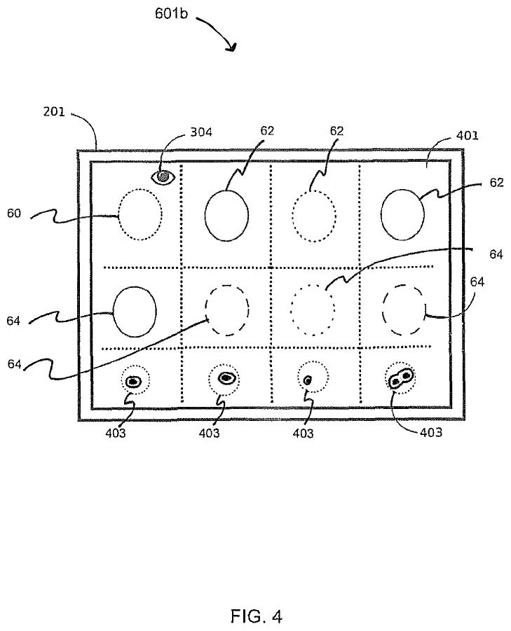

FIG. 4 depicts the alternate views workflow 601b which preferably enables the user 210 to access and view alternate and/or additional images which may aid in the interpretation of the desired imaging data 60. In preferable embodiments, the system 50 includes a hanging protocol 401, which includes a series of images based on alternate data 62 (alternatively “other data 62”), such as alternate images, obtained at or about the same time as the desired current imaging data 60. Persons skilled in the art will understand that a hanging protocol is the series of actions performed to arrange images for optimal viewing to facilitate the presentation of specific types of studies, for example, in a consistent manner and/or to reduce the number of manual image ordering adjustments performed by the user. The hanging protocol 401 is preferably adapted to additionally include preselected or predetermined instances of any desired imaging data 60, such as preselected viewing settings, orientations and/or regions of interest. Additionally, the alternate views workflow 601b is preferably adapted to include an area for the viewing and retrieval of prior imaging data 64 from the database 220 (also shown in FIG. 2). Prior imaging data 64 preferably includes imaging data from the same patient acquired previously, from another type of imaging modality than the current one and/or from any imaging data 60 which may be of some relevance and/or interest, such as data which has a clinically relevant presentation to the current data and stored in a local and/or remote database 220. Another component which is preferably included in the alternate views workflow 601b is the presentation and retrieval of a prior CAD output 403. In a manner similar to the prior imaging data 64, the prior CAD output 403 is preferably any previously generated CAD output, or any CAD output currently generated from prior imaging data 64. In preferable embodiments of the present invention, a user 210 may interact with each component of the alternate views workflow 601b via the biometric interaction system 101. Preferably, the biometric interaction system 101 enables the user 210 to manipulate and/or interact with (e.g., select, retrieve, magnify, compare, navigate and/or enable) any other function with respect to image data 60 that a person skilled in the art may expect within the radiology workflow environment 100 using one or more peripherals 205 (e.g., the eye-tracking device 205d and/or the motion-tracking device 205e).

FIG. 5 depicts a reporting workflow 601d in accordance with a preferred embodiment. The workflow 601d is preferably adapted to include components to facilitate the recording of the interpretation by the user 210 of the image data 60 and the generation of a record of the interpretation of the data. In a preferred embodiment of the present invention, the reporting workflow 601d includes a structured report 501 and interactable fields 503 mediated by the biometric interaction system 101 as displayed on one or more secondary workflow displays 202. In preferable embodiments, an active field 502 is one which is understood to be currently interactable to the user 210. In an embodiment of the present invention, indicator 304 is preferably used to convey the status of an active field 502 (e.g., eye gaze). In preferable embodiments of the present invention, the interactable fields 503 of the workflow 601d are applied to adjust, modify and/or annotate the contents in the active field 502. The interactable fields 503 preferably include, for example, text fields, selectable options, selectable grades and/or rankings. Interactable fields 503 are preferably optimized for use with the biometric interaction system 101 instead of the traditional user interfaces designed for standard peripherals such as a keyboard of mouse.

In accordance with a preferred embodiment, a worklist workflow 601e is shown in FIG. 6 in association with the one or more secondary imaging displays 202. The workflow 601e includes components that enable interactions with other data 62 the user 210 may interact with, such as other cases needing interpretation (the “worklist queue”) or prior data that are relevant to the current case (“worklist priors”). In a preferred embodiment of the present invention, the worklist workflow 601e includes one or more selection panes 602 which may be configured to preview information relevant to either the worklist queue or the worklist priors. The one or more selection panes 602 are preferably adapted to include interactable elements as enabled by the biometric interaction system 101. The worklist workflow 601e may also include a preview pane 603 adapted to preview image data 60 or other data 62 (including text data 303, peripheral data 230) and may also be mediated by the biometric interaction system 101. It is understood that the purpose of the worklist workflow 601e is to allow the user 210 to seamlessly interact with data from other cases available in the worklist queue or the worklist priors. In an embodiment of the present invention, the indicator 304 may preferably be used to help convey the status (e.g., eye gaze), such as selection status, of any interactable elements.

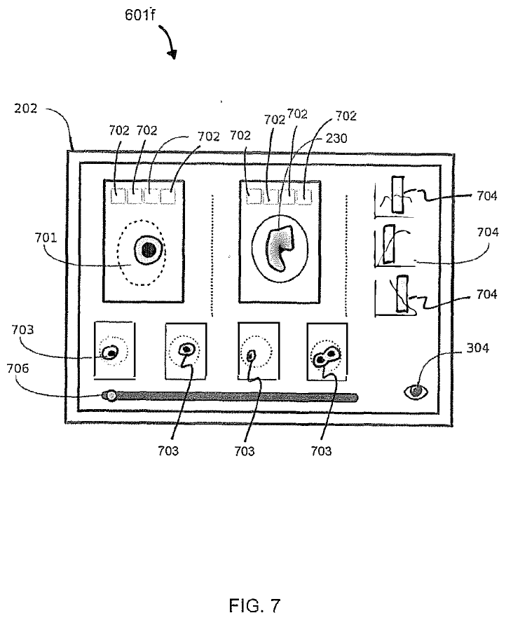

FIG. 7 depicts a CAD workflow 601f in accordance with a preferred embodiment displayed on the one or more secondary imaging displays 202. The workflow 601f preferably includes a single viewing pane 301, or a plurality of panes displaying interactable components within a GUI or the one or more secondary imaging displays 202. In accordance with a preferred embodiment, such components may include a CAD output 701 which preferably displays results or other information associated with the image data 60 based on the application of one or more CAD algorithms using the processor(s) 203, 204, including: a CAD querying component 702 which is preferably adapted to facilitate a request by the user 210 and/or retrieve specific types of information from the CAD system 102 which may, for example, be stored on a local and/or remote database 220; alternate CAD outputs 703, which is preferably adapted to display the CAD output of alternate views (or alternate data 62) or prior imaging data 64; the presentation of population metrics 704 and peripheral data 230 (including, for example, gaze analysis information); and a CAD output stream 706 to facilitate rapid and/or simple toggling or scrolling of multiple CAD outputs 701 by the user 210. In accordance with a preferable embodiment, each one of the CAD workflow components are interactable through the biometric interaction system 101. In a preferred embodiment, however, one or more functions made available by the biometric interaction system 101 are specific to the CAD workflow 601f including, for example, peripheral data 230 collected by the peripherals 205 (e.g., data collected by the eye-tracking device 205d) which may include information about the cognitive attention of the user 210 which may be cross-referenced by the processors 203, 204 with information made available by the CAD system 102. In a preferred embodiment, the peripheral data 230 (e.g., including cognitive attention) may be used to alert the user to missed diagnosis/diagnoses (for example) based on the CAD output 701. For example, the peripheral data 230 collected by the eye-tracking device 205d may preferably be used to alert the user if the CAD output 701 has not been viewed.

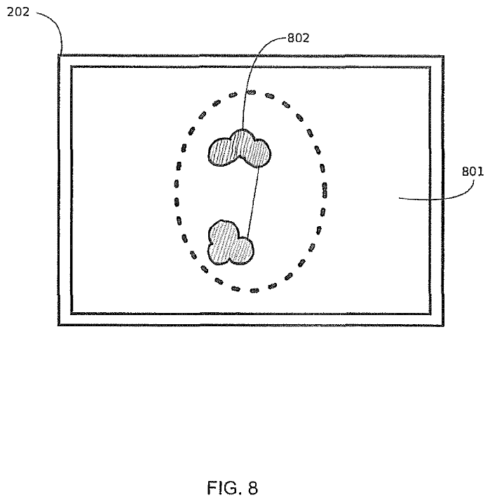

FIG. 8 depicts an imaging bookmark 802 in accordance with a preferred embodiment of the present invention. In a preferable embodiment, the biometric interaction system 101 may detect an interruption which takes a user's attention away from the radiological workflow environment 100 and/or the CAD system 102. The biometric interaction system 101 preferably detects when the user 210 returns their attention to the previous task. By collecting and/or storing information about the user's activities, such as gaze patterns (e.g., using the peripheral data 230), before the interruption and making available graphical, auditory and/or other features which are indicative of the user's prior state of attention, the effect of the interruption may be mitigated. In accordance with a preferred embodiment of the present invention, one such example may be the implementation of an imaging bookmark 802 within a viewing area 801 to indicate to the user 210 which areas of an image or graphical user interface 201, 202 their attention was focused on before the interruption.

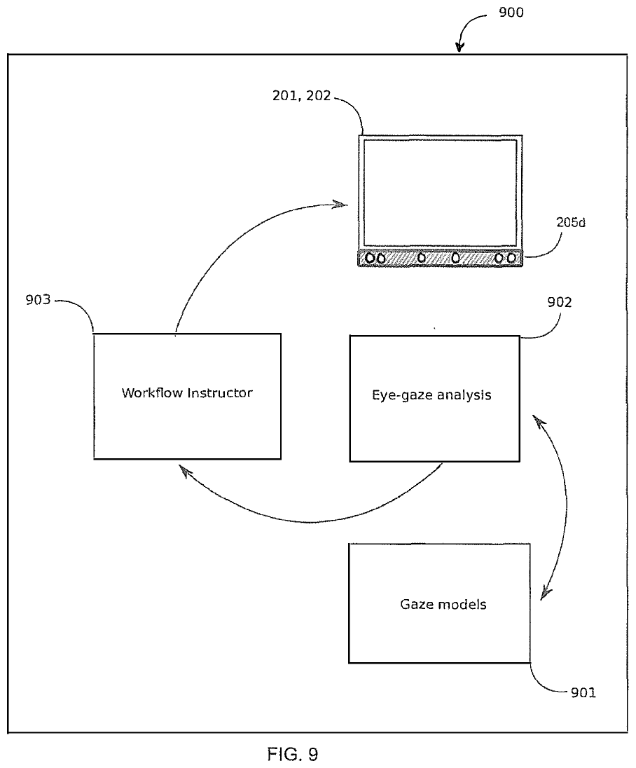

In a preferred embodiment, the workflow 601 may be combined with the biometric interaction system 101 in a number of ways to preferably facilitate additional functionalities. As shown in FIG. 9, methods for peripheral data analysis 900 (e.g., analysis of peripheral data 230 including gaze data collected by the eye-tracking device 205d) preferably enable these additional functionalities such as gaze analysis. In preferable embodiments, features which may facilitate this enablement include one or more trained gaze models 901 adapted to be either user specific or not user specific, an eye tracking analysis module 902 and a workflow instructor 903. The one or more trained gaze model 901 (or models 901) are preferably trained using machine learning, a rule-based algorithm or other methods known to persons skilled in the art (e.g., third-party data sets). In preferred embodiments, the processor 203, 204 is adapted to apply the one or more trained gaze models 901 to the imaging data 60 to identify and send notable patterns, sequences and/or other combinations of data (from which higher order information may be inferred, such as recognition and/or attention of the user) to the eye-tracking analysis module 902 and the workflow instructor 903. The eye tracking analysis module 902 is preferably adapted to receive data 230 from the eye-tracking device 205d and processor(s) 203, 204 associated with the system 50 and apply an eye tracking analysis module algorithm to interpret the data 230 in the context of the interactable features currently in use, any information or output made available by the CAD system 102, previous actions made by the user 210, and/or any other feature or action relevant to the purposes of viewing and interpreting image data 60 including medical images. The workflow instructor 903 is preferably adapted to initiate specific actions based on either of the trained gaze model(s) 901 and/or the eye tracking analysis module 902 using, for example, the processor(s) 203, 204 associated with the system 50. In accordance with a preferred embodiment of the present invention, the trained eye gaze models 901, the eye tracking analysis module 902, and the workflow instructor 903, can be implemented within the same computer system, within the same software or computer application, within the primary imaging display 201, and/or the secondary imaging display 202 or in any combination thereof.

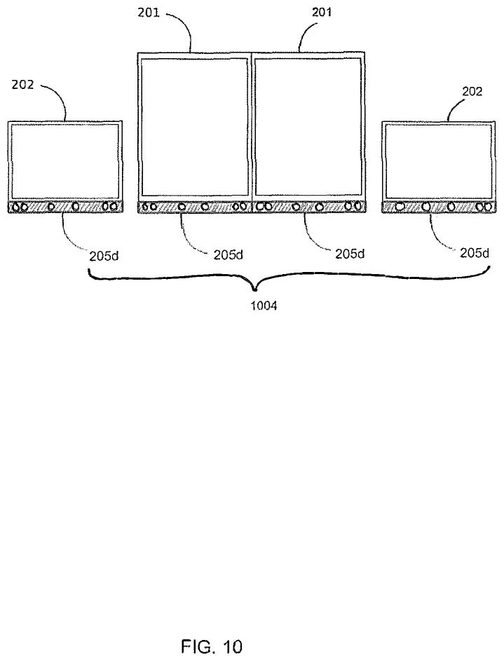

In a preferred embodiment, peripherals 205 may be adapted to form an array of multiple components or interdependent devices 1004. For example, as shown in FIG. 10, multiple eye-tracking devices 205d are operatively connected to form an array of interdependent devices 1004 that may be adapted to operate as a single combined device or one or more combinations of independent devices in association with one or more primary imaging displays 201 and/or one or more secondary imaging displays 202. The data from the array of interdependent devices 1004 is preferably aggregated and/or harmonized to determine and/or record the peripheral data 230 including the gaze information as used and stored by other components of the system 50 (e.g., the database 220). The array of interdependent devices 1004 may, in certain embodiments, have the ability to serve as an eye-tracking device (as depicted in FIG. 10).

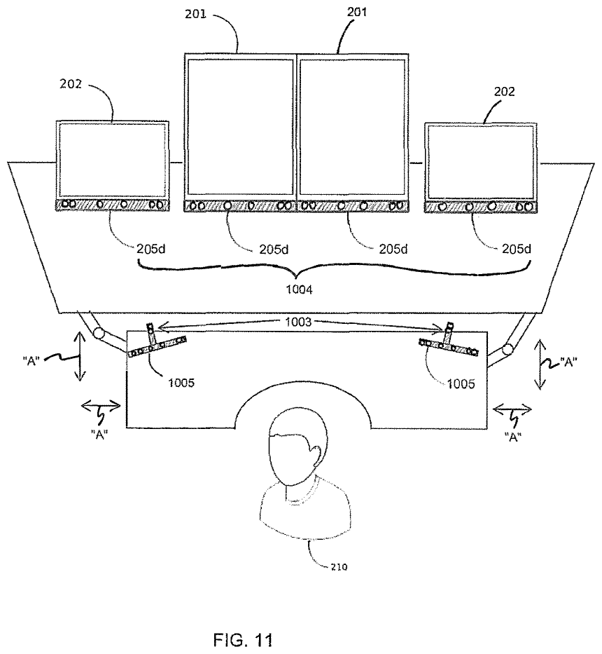

In an another embodiment of the present invention, the eye-tracking device 205d or array of interdependent devices 1004 (for eye-tracking as depicted in FIG. 11) may include one or more peripherals that are mechanically separated (as depicted by the “A” arrows) from the primary imaging display 201 and/or the secondary imaging display 202 as shown in FIG. 11. This mechanical separation may be such that the eye-tracking device 205d or at least a portion of the interdependent devices 1004 may be relocated independently from the displays 201,202 to accommodate the position of the user 210 for example (e.g., increase eye tracking accuracy). One or more supplementary tracking markers 1003 are preferably used to determine and/or record the location of the supplementary tracking element 1005 (i.e., the mechanically separated eye-tracking device) relative to the array of interdependent devices 1004 (which are preferably screen mounted) to facilitate the determination of the eye location of the user 210. Together, the supplementary tracking markers 1003 and supplementary tracking elements 1005 preferably form a cascade of instrumentation which preferably aggregates and/or harmonizes the data obtained to calculate and/or record the peripheral data 230 (e.g., gaze data) as used and/or stored by other components (e.g., database 220) in accordance with one or more embodiments of the present invention. Supplementary tracking elements 1005 may in certain embodiments have the ability to serve as an eye-tracking device 205d or as an additional component of the array of interdependent devices 1004 which are adapted to measure eye location relative to itself.

In another embodiment of the present invention, the eye tracking device 205d, array of interdependent devices 1004, and/or the supplementary tracking elements 1003, are managed by the workflow instructor 903 (shown in FIG. 9), which is preferably adapted to synchronize and/or coordinate their functions via the eye-tracking analysis module 902, the biometric interaction system 101, and/or the processor(s) 203, 204 included in the workflow environment 100 to preferably optimize data quality and accuracy (e.g., avoiding interference between devices or device components). In certain preferable embodiments, the effective field of view of the aforementioned tracking devices is restricted, either manually or automatically, for example, by physical means to facilitate optimization of data quality and/or accuracy.

In an embodiment of the present invention, the eye gaze analysis module 902 (alternately “eye-tracking analysis module 902”) is preferably adapted to detect when the user 210 is interrupted during the task of viewing and/or interpreting data including image data 60, or the CAD output 102 derived from such data. In this manner, through the workflow instructor 903, the system 50 preferably creates new workflows 601 (e.g., an alert workflow; not shown) to call attention to the state of the workflow 601 prior to the interruption. For example, graphical features may be overlaid onto the imaging pane 301 to mitigate the effect of the interruption on data viewing and/or interpretation.

In another embodiment of the present invention, the biometric interaction system 101 preferably detects the absence of the user 210 in a position to interact and/or view the radiological workflow environment 100. A digital privacy screen (not shown) is preferably initiated by the workflow instructor 903 to reduce or eliminate the potential for a privacy breach by, for example, blurring the primary imaging display 201 and/or the secondary imaging display 202, including disabling the displays 201, 202 or otherwise obfuscating any information that would have otherwise remained on the displays 201, 202. In preferable embodiments, the biometric interaction system 101 is reactivated when the user 210 (or any other authorized user) returns to the position of interacting with the radiological workflow environment 100; the digital privacy screen is deactivated, and the workstation is returned to the previous state, or an augmented state, to mitigate the effect of the absence of the user.

In another embodiment of the present invention, the biometric interaction system 101 and/or the eye gaze tracking and analysis module 902 is preferably adapted for use in conjunction with modified versions of the current image data 60, such as edge maps, gradient images, saliency maps and/or maps of feature sets. In yet another embodiment of the present invention, the workflow instructor 903 is preferably adapted to communicate with databases external to the system 50 to retrieve anatomical information (e.g., atlases, models and/or classifications algorithms) to orchestrate a workflow including an anatomical context.

In another embodiment of the present invention, the eye gaze tracking and analysis module 902 and/or the biometric interaction system 101 is preferably adapted to store in the database 220 and/or analyze longitudinal data from the user 210 to derive indicators of performance, detect biases, and/or provide other higher-level information about the image interpretation by the user 210.

In another embodiment of the present invention, the eye gaze tracking and analysis module 902 and/or the biometric interaction system 101 is preferably adapted to store in the database 220 and/or analyze data from a set or group of users to derive indicators of performance, detect biases and/or provide other higher-level information about the image interpretation by the group of users, and/or to individual users within the group.

In another embodiment of the present invention, the eye gaze tracking and analysis module 902 and/or the biometric interaction system 101 is preferably adapted to store in the database 220 and/or analyze data which relates to one or more specific workstations (or radiology workflow environment 100), preferably comprising their physical characteristics and/or any software or applications that are also part of the user's workflow and working environment.

In another embodiment of the present invention, visual feedback is presented to the user in a temporally relevant manner. In certain embodiments, this may preferably include an immediate alert: (i) while reading an image; (ii) immediately before reading an image is expected to conclude; (iii) once an image is finished being read, or (iv) any other span of time relevant to functions being performed by the user. The visual feedback may bring to the attention of the user 210 any relevant or critical information obtained from information gleaned by the eye gaze tracking and analysis module 902 and/or the biometric interaction system 101. The visual feedback may be presented on one or more primary imaging displays 201 and/or one or more secondary imaging displays 202 and preferably overlaid onto the relevant medical imaging information as seen and/or interpreted by the user 210. In another embodiment of the present invention, the visual feedback may be presented using color-coding, textures, lines, shapes, or any other design and in a consistent manner so that the information being presented may be quickly assimilated by the user. In another embodiment of the present invention, visual feedback includes peripheral data 230 from the biometric interaction system 101, including eye gaze tracking data, that is aggregated from one or more imaging displays 201, 202 or areas within these displays. The visual feedback may aggregate information from multiple views of the same anatomical region, acquired at different time points, acquisition protocols, or other imaging modalities including both two-dimensional, three-dimensional and time series imaging data. As an example, peripheral data 230 including eye gaze tracking data obtained during the viewing of a three-dimensional digital breast tomosynthesis is aggregated and presented onto standard or synthesized two-dimensional mammographic images.

In another embodiment of the present invention, peripheral data 230 (e.g., eye gaze data) from the biometric interaction system 101, the eye gaze tracking and analysis module 902, and/or any other software or application component of the workflow environment 100 are preferably used to reconcile eye gaze data with image modifications as performed and/or seen by the user. Such image modifications may include zooming, panning, scrolling and/or any other manipulation that may alter the images on the screen. In another embodiment of the present invention, image registration and/or transformation algorithms may be used to reconcile the eye gaze data to the image modifications. In another embodiment of the present invention, input from the user 210 may be used to keep track of the image modifications performed. In yet another embodiment of the present invention, the image modifications are detected without knowledge of user action through the use of image registration and/or transformation algorithms and monitoring image output on any of the displays. In yet another embodiment of the present invention, the use of prior anatomical information or modelling is used to aid the reconciliation of the eye gaze data. In another embodiment of the present invention, the image modifications comprise, consist essentially, or consist of navigating through a three-dimensional image set or “stack”.

In another embodiment of the present information, data from the biometric interaction system 101 and/or the eye gaze tracking and analysis module 902 are preferably adapted for use to generate predictions of user input into the structured report 501 or into any given component of the structured report 501, such as an active field 502 and/or interactable field 503.

In an embodiment of the present invention, the biometric interaction system 101 preferably reduces the amount of (at least a portion or preferably significant) manual input from the user 210 using traditional peripherals, such as a keyboard 205a or mouse 205b. There may be multiple principal mechanisms by which the use of manual input may preferably be reduced or eliminated.

In preferable embodiments of the present invention, the system 50 is adapted to include data 230 collected by one or more components of the biometric interaction system 101, such as the eye-tracking device 205d and/or the motion tracking device 205e (including gesture tracking), which preferably reduces or eliminates manual input by the user. For instance, selecting a graphical user interface item on a display 201, 202 may preferably be replaced by aspects of the gaze of the user 210, such as a fixation, blinking, dwell time and/or head movement. Further, an action formerly mediated by the computer mouse 205b either by using a scroll bar on the display or a physical scroll wheel provided by the mouse 205b may preferably be replaced by a hand gesture that is detected by the motion tracking device 205e. Preferably, the direction of the motion (e.g., a gesture), including the specific hand and/or finger positions of the gesture, facilitate operation of the functions along multiple dimensions. Persons of ordinary skill in the art may appreciate that a minority or majority of functions formerly performed with a keyboard 205a and mouse 205b may be replaced by the functionality enabled by the biometric interaction system 101 and that the preferences of the user 210 and methods to set these preferences within the biometric interaction system 101 are also part of the present invention.

In preferable embodiments of the present invention, the system 50 is adapted for the biometric interaction system 101 to reduce the manual input required of the user 210 by instructing workflow context to the radiological workflow environment 100 and/or the CAD system 102. For example, it may be common within most graphical user interfaces for only a single item or group of items visible on the display to be in focus, and only the item in focus can be intractable with the keyboard 205a and mouse 205b. A text field within the reporting workflow 601d may not have text entered into it via the keyboard 205a or the voice-operated device 205c without having been manually selected first via the keyboard 205a or mouse 205b. In a preferable embodiment, the biometric interaction system 101 enables various items to be brought into focus based on data collected by the eye-tracking device 205d and/or the motion tracking device 205e and analyzed by the gaze analysis module 900 and/or a processor 203, 204 associated with the system 50. Persons of ordinary skill in the art may appreciate that the impact of the workflow items being automatically brought into focus for the user based on the function of the biometric interaction system 101 is preferably greater in the case of a radiological workflow environment with a plurality of computer displays of possibly non-uniform sizes and/or orientation.

In a preferred embodiment, a workflow context generated by the biometric interaction system 101 is adapted to further leverage known or trained workflow strategies to guide the user 210 within the workflow 601. These workflow strategies reference the trained eye gaze model 901. Further, in another embodiment of the present invention, the results from the CAD system 102 further instruct the workflow strategy. An example would be for an area of suspicion, as detected by the CAD system 102 and for which the biometric interaction system 101 may indicate the user 210 has either recognized or failed to consciously acknowledge the area of suspicion. The biometric interaction system 101 may also infer for the information collected whether the area of suspicion was recognized as belonging to a certain class.

In a preferred embodiment, the workflow context may also mediate different paradigms under which information from the CAD system 102 may be made available to the user 210. For example, information from the CAD system 102 is preferably presented concurrently to the user 210, within a second reader paradigm or upon a query initiated by some action of the user 210, whether explicitly through some voluntary action, or indirectly as mediated by the biometric interaction system 101.

In a preferred embodiment, the biometric interaction system 101 is also adapted for use within the radiology workflow environment 100, with or without the CAD system 102 to provide feedback to a user 210 or plurality of users 210 in an educational or training setting or as a method of quality assurance. This feedback may be immediate, delayed, stored in the database 220 or aggregated for review by the user or some other concerned party.

In a preferred embodiment, gaze models 903 for a new user 210 are initialized using gaze models which already exist created for other, prior users using transfer learning or other knowledge or data initialization techniques.

In a preferred embodiment, the primary 201 and/or secondary 202 displays perform all functions, or a subset of functions of the present invention. The primary 201 and or secondary 202 displays may physically encapsulate any and all software and/or hardware required to perform those functions.

In accordance with a preferred embodiment of the present invention, the use of the radiological workflow environment with the CAD system may preferably be further harmonized by the biometric interaction system through the implementation of models of saliency (e.g., to predict eye movements made during image viewing without a specified task or free viewing) and suitability (weights locations relative to each other based on predetermined criteria) within the workflow instructor. In a preferred embodiment, measures of the salience and suitability of particular image analysis algorithms are provided by the user, either consciously or unconsciously. An example of this, using an eye-tracking device and an eye-gaze tracking and analysis module would be the monitoring of gaze dwell times, number of fixations and/or pupil dilations. In preferred embodiments of the present invention, these measures of saliency and suitability may be combined to those provided by the algorithms themselves by the workflow instructor.

In accordance with a preferred embodiment of the present invention, the biometric interaction system may improve the ergonomics of the work performed by radiologists by for example reducing the volume of clicking and scrolling with a standard computer mouse. In an embodiment, the present invention preferably reduces the incidence of repetitive strain injuries such as tendonitis. In accordance with a preferred embodiment of the present invention, the biometric interaction system may be readily adaptable to multiple users within a single clinical environment. Therefore, unlike the traditional approach of improving ergonomics by interchanging physical items (e.g., desks and chairs), the present invention may result in a higher uptake by radiologists as the physical barriers to adapt or customize the features of the biometric interaction system to individual users may be reduced.

In accordance with a preferred embodiment of the present invention, there may be provided additional context that can be brought to any feature of the radiology workflow. In a preferred embodiment, for example, radiology reports are produced by radiologists as they read the image where they may remark on specific organs or features. The biometric interaction system, particularly the gaze-tracking and analysis module, may preferably be adapted to instruct the radiology reporting components of the system as to the user's intention for reporting. In a preferred embodiment, for example, fields for a particular organ may be populated automatically after a radiologist looks at an organ and carries their gaze towards the reporting component of the radiological workflow. Additionally, the reporting component of the radiology workflow may also be responsible for a significant portion of manual user interaction which burden radiologists. In a preferred embodiment, features of the reporting component may preferably but need not necessarily be activated, initiated and/or rendered interactable by the workflow instructor aided by information from the eye gaze tracking and analysis module. In a preferred embodiment of the current invention, the reporting component is a structured report.

In accordance with one or more preferred embodiments, the system, method and/or computer readable medium of the present invention may provide alerts and/or other information to a radiologist, including but not limited to alerts relating to a missed diagnosis and/or other lapses in visual attention. Preferably, the generation of such alerts and/or other information would include the analysis of patterns in gaze data which are found to be a fit or a misfit to particular known gaze patterns. Such generation of alerts and/or other may further involve in certain embodiments the combination of CAD information with the gaze information.

In accordance with one or more preferred embodiments, the system, method and/or computer readable medium of the present invention may provide gaze tracking implemented for a radiology workflow environment which may preferably include multiple viewing displays spanning a large area and/or large volume within which to track the user's gaze.

In a preferred embodiment, any or all of the elements presented may be implemented in an agnostic manner, such that software or application elements of the radiology workflow environment do not make available any information and/or data, but that this data is collected via capture of the primary and/or secondary display output and/or any other peripherals present in the workflow environment.

### Data Store

A preferred embodiment of the present invention provides a system comprising data storage (e.g., databases) that may be used to store all necessary data required for the operation of the system. A person skilled in the relevant art may understand that a “data store” refers to a repository for temporarily or persistently storing and managing collections of data which include not just repositories like databases (a series of bytes that may be managed by a database management system (DBMS)), but also simpler store types such as simple files, emails, etc. A data store in accordance with the present invention may be one or more databases, co-located or distributed geographically or cloud based. The data being stored may be in any format that may be applicable to the data itself, but the data may also be in a format that also encapsulates the data quality.

The foregoing description has been presented for the purpose of illustration and may not be intended to be exhaustive or to limit the invention to the precise form disclosed. Other modifications, variations and alterations are possible in light of the above teaching and may be apparent to those skilled in the art, and may be used in the design and manufacture of other embodiments according to the present invention without departing from the spirit and scope of the invention. It may be intended that the scope of the invention be limited not by this description but only by the claims forming a part of this application and/or any patent issuing therefrom.

1. A method for collecting and providing a user's visual feedback to image data in a workflow environment comprising an imaging display for presenting content to the user, the method comprising:
(a) operating a biometric interaction system comprising (i) a motion tracking device that receives motion data associated with a movement of the user; (ii) an eye-tracking device that receives gaze data associated with an eye gaze of the user; and (iii) a peripheral processor that collects and transmits the motion data and the eye gaze data; and
(b) operating a computer-aided detection or diagnosis (“CAD”) system comprising a system processor to: (i) electronically receive the motion data and the gaze data from the peripheral processor; (ii) analyze the image data using a computer-aided diagnosis algorithm to automatically identify a feature associated with the image data; (iii) present the image data, the identified feature, or both thereof to the user on the imaging display; and (iv) automatically apply the motion data to the imaging display using a gesture algorithm and the gaze data to the imaging display using an eye tracking analysis module algorithm to manipulate the content of the imaging display.

2. The method of claim 1, wherein the imaging display comprises a primary imaging display and a secondary imaging display.

3. The method of claim 2, further comprising a step of electronically storing the motion data, the gaze data, the image data, the identified feature, or a combination of any or all thereof in a database.

4. The method of claim 3, further comprising a step of applying one or more predetermined workflows to facilitate the provision of visual feedback of the image data by the user.

5. The method of claim 4, wherein the one or more predetermined workflows comprise an imaging workflow, an alternate views workflow, a reporting workflow, a worklist workflow, a CAD workflow, or a combination of any or all thereof.

6. The method of claim 5, wherein the biometric interaction system further comprises an array of interdependent devices.

7. The method of claim 6, wherein the array of interdependent devices comprises two or more eye-tracking devices.

8. The method of claim 7, wherein the imaging display comprises anatomical data and the method detectably improves the diagnostic value of the anatomical data.

9. The method of claim 8, wherein the one or more imaging workflows comprise radiological workflows.

10. The method of claim 8, wherein the imaging data comprises multiple views of the same anatomical region.

11. The method of claim 8, wherein the imaging display comprises both two-dimensional and three-dimensional time series imaging data.

12. A system for collecting and displaying visual feedback to image data presented to a user, comprising:
(a) a workflow environment comprising an imaging display for presenting content to a user;
(b) a biometric interaction system operative to facilitate interaction with the imaging display by the user, comprising: (i) a motion tracking device adapted to receive motion data associated with a movement of the user; (ii) an eye-tracking device adapted to receive gaze data associated with an eye gaze of the user; and (iii) a peripheral processor operative to collect and transmit the motion data and the eye gaze data; and
(c) a computer-aided diagnosis system comprising a system processor operative to: (i) electronically receive the motion data and the gaze data from the peripheral processor; (ii) analyze image data using a computer-aided diagnosis algorithm to automatically identify a feature associated with the image data; (iii) present the image data, an identified feature, or both thereof, to the user on the imaging display; and (iv) manipulate the content of the imaging display by automatically applying (I) the motion data to the imaging display using a gesture algorithm and (II) the gaze data to the imaging display using an eye tracking analysis module algorithm to manipulate the content.

13. The system of claim 12, wherein the imaging display comprises a primary imaging display and a secondary imaging display.

14. The system of claim 13, the system further comprising a database to electronically store the motion data, the gaze data, the image data, the identified feature, or a combination of any or all thereof.

15. The system of claim 14, further comprising one or more predetermined workflows to facilitate the provision of visual feedback of the image data by the user.

16. The system of claim 15, wherein the predetermined workflows comprise: an imaging workflow, an alternate views workflow, a reporting workflow, a worklist workflow, a CAD workflow, or a combination of any or all thereof.

17. The system of claim 16, wherein the biometric interaction system further comprises an array of interdependent devices.

18. The system of claim 17, wherein the array of interdependent devices comprises two or more eye-tracking devices.

19. A non-transitory computer readable medium on which is physically stored executable instructions, which upon execution provide visual feedback of a user to image data presented to the user within a workflow environment comprising an imaging display for presenting content to the user, a biometric interaction system comprising a motion tracking device adapted to receive motion data of the user and an eye-tracking device adapted to receive gaze data of the user to facilitate interaction with the imaging display by the user and a computer-aided diagnosis system, wherein the executable instructions comprise processor instructions for a peripheral processor and/or a system processor to automatically:
(a) collect and/or electronically communicate the motion data from the peripheral processor to the system processor;
(b) collect and/or electronically communicate the gaze data from the peripheral processor to the system processor;
(c) automatically identify a feature associated with the image data using a computer-aided diagnosis algorithm;
(d) automatically present the image data and/or the identified feature to the user on the imaging display; and
(e) automatically manipulate the content of the imaging display using a gesture algorithm on the motion data and the eye tracking analysis module algorithm on the gaze data.

20. A system comprising at least one processor and the non-transitory computer readable medium of claim 19.
